(19) United States
(12) Patent Application Publication
(10) Pub. No.: US 2007/0219481 A1
(43) Pub. Date: Sep. 20, 2007

(54) Apparatus and methods for the treatment of avian influenza with ultrasound

(76) Inventor: Eilaz Babaev, Minnetonka, MN (US)

Correspondence Address: EILAZ BABAEV, 5929 BAKER ROAD, SUITE 470, MINNETONKA, MN, 55345, US

(21) Appl. No.: 11/384,155

(22) Filed: Mar. 16, 2006

(51) Int. Cl. A61B 17/20

(52) U.S. Cl. 604/22; 601/2

(57) ABSTRACT

The method and device of the present invention for the treatment of avian influenza and other viruses, bacteria, and infectious agents with ultrasonic waves includes a generator and a transducer to produce ultrasonic waves. The ultrasonic transducer has a specially designed ultrasound tip that is able to radiate ultrasound energy toward the body of the target animal or human at a level higher than a traditional ultrasound tip. The apparatus delivers ultrasonic energy without contacting the target through an air/gas medium or through a spray, or by contacting the target with or without a coupling medium. The ultrasonic waves provide a therapeutic effect such as destroying viruses, bacteria, or other infectious agents, increasing blood flow, or stimulating the immune system, etc; additionally, breathing ability is enhanced through the ultrasonic delivery of a medicament to the target's lungs.

### BACKGROUND OF THE INVENTION

1. Field of the Invention

The present invention relates to the treatment of avian influenza with ultrasound. In particularly, the present invention relates to an apparatus and methods using ultrasonic energy for the treatment of humans and birds infected with an avian influenza virus.

2. Description of the Related Art

“Avian influenza (AI) or Fowl Plague is a disease of viral etiology that ranges from a mild or even asymptomatic infection to an acute, fatal disease of chickens, turkeys, guinea fowls, and other avian species, especially migratory waterfowl.

Fowl plague was described in 1878 as a serious disease of chickens in Italy. It was determined in 1955 that fowl plague (FP) virus is actually one of the influenza viruses. The AI viruses, along with the other influenza viruses, make up the virus family Orthomyxoviridae. The virus particle has an envelope with glycoprotein projections with hemagglutinating and neuraminidase activity. These two surface antigens, hemagglutinin (HA) and neuraminidase (NA), are the basis of describing the serologic identity of the influenza viruses using the letters H and N with the appropriate numbers in the virus designation e.g., H7N2. There are now 15 hemagglutinin and 9 neuraminidase antigens described among the Type A influenza viruses. The type designation (A, B, or C) is based upon the antigenic character of the M protein of the virus envelope and the nucleoprotein within the virus particle. All influenza viruses affecting domestic animals (equine, swine, avian) belong to Type A, and Type A influenza virus is the most common type producing serious epidemics in humans.” (Gray Book. Foreign Animal Diseases. Part IV. 1998 edition. Available online at: http://www.vet.uga.edu/vpp/gray_book/FAD/avi.htm)

The avian virus causes two distinct forms of disease—one that is common and mild, and the other that is rare and highly lethal. The version that has gained recent public exposure is the highly pathogenic H5N1 influenza A virus that occurs mainly in birds and is extremely deadly. Its effects on poultry are dramatic—it spreads rapidly and has a mortality rate that can approach 100%, and death often occurs within 48 hours.

Even though H5N1 is an avian virus, it has been known to infect humans from either direct or close contact with infected poultry or contaminated surfaces. Only a few rare cases have occurred where the virus spread from one person to another, though the infection has not continued beyond one person. There have been recent confirmed human cases of the H5N1 virus in Cambodia, China, Indonesia, Iraq, Thailand, Turkey, and Vietnam.

There is currently no known effective treatment for H5N1 in humans. The recent isolates are highly resistant to amantadine and rimantadine, two antiviral medicines commonly used for influenza; therefore, those drugs will not have a therapeutic role in treating Avian Influenza. There are limited data that oseltamavir (Tamiflu) and zanamvir (Relenza) may be effective against the H5N1 virus. Antiviral resistance to these drugs has been minimal so far, but drug resistant virus strains are likely to develop if there is a worldwide outbreak.

The concern with the H5N1 virus is that it will alter form and gain the ability to spread easily from one person to another. Because these viruses do not commonly infect humans, the general population has little, if any, natural immune protection. Therefore, a worldwide outbreak of the disease could begin if it becomes contagious among humans. The virus is deadly in humans; according to the World Health Organization (WHO), as of Mar. 6, 2006, there have been 175 reported cases with over half resulting in death. Available online at: http://www.who.int/csr/disease/avian_influenza/country/cases_table_{—}2006_{—}03_{—}06/en/index.html Furthermore, WHO claims that the result of a worldwide outbreak would, under the best circumstances and assuming that the new virus causes only a mild disease, result in an estimated 2 million to 7.4 million deaths. Available online at: http://www.who.int/csr/disease/avian_influenza/avian_faqs/en/index.html

Another problem with viruses is that they cause infections that force the immune system to respond, thus decreasing the body's ability to protect itself from other bacterial and viral infections. Furthermore, viral infections can result in a weakened immune system. Even if the virus is treated, the body's immune system still must be strengthened and restored to its normal level.

Therefore, a need exists for an apparatus and methods that can effectively treat avian influenza where the virus cannot adopt a resistance and that can also stimulate the body's immune system back to its normal level.

Ultrasonic waves are well known for their use in medical diagnostic and therapeutic applications as well as for their use in many industrial applications. One diagnostic use of ultrasound waves is ultrasonic imaging—this process includes using ultrasonic waves to detect underlying structures in human tissue. Imaging is conducted by placing an ultrasonic transducer in contact with the tissue via a coupling medium, and then high frequency (1-20 MHz) ultrasonic waves are directed into the tissue. The ultrasonic waves are reflected back to a receiver adjacent to the transducer after contact with underlying structure. An image of the underlying structure is produced by comparing the signals of an ultrasonic wave that is sent to the signals of the reflected ultrasonic wave that is received. This technique is particularly useful for identifying boundaries between components of tissue and can be used to detect irregular masses, tumors, and the like.

Two therapeutic medical uses of ultrasonic waves are aerosol mist production and contact physiotherapy.

Aerosol mist production makes use of a nebulizer or an inhaler to produce an aerosol mist for creating a humid environment and for delivering drugs to the lungs. Ultrasonic nebulizers direct ultrasonic waves through a liquid and towards an air-liquid interface from a point either underneath or within the liquid. The ultrasonic waves disintegrate capillary waves, thus causing liquid particles to eject from the surface of the liquid into the surrounding air. This technique can produce a very fine dense fog or mist. Ultrasound is the preferred method to produce aerosol mists because ultrasonic waves allow for the production of a smaller aerosol particle size. One of the major shortcomings of inhalers and nebulizers, however, is that an air stream is necessary to direct the aerosol particles towards the target, thus decreasing the efficiency of the ultrasound.

Contact physiotherapy attempts to produce a physical change in tissue by directly applying ultrasonic waves. Generally, an ultrasonic transducer contacts the tissue via a coupling medium. Ultrasonic waves produced by the transducer travel through the coupling medium and into the tissue. Examples of coupling mediums are a bath of liquid, a jelly applied to the surface to be treated, a water-filled balloon, a gel, and a gel pad. Conventional techniques use ultrasonic waves that have an intensity of from about 0.1 w/cm² to 3 w/cm² at a frequency of from about 0.8 to 3 Megahertz. The treatment is applied to a skin surface for about 1 to 30 minutes, two or three times a week. The coupling medium can provide a cooling effect by dissipating some of the heat energy that is produced by the ultrasonic transducer. Because ambient air is a relatively poor medium for the propagation of ultrasonic waves, the traditional manner in which to transmit the ultrasonic waves from the transducer to the skin surface was to use either a coupling medium or a direct contact between the tissue and the ultrasonic transducer.

Contact ultrasound physiotherapy provides several beneficial effects, including local improvement of blood circulation, tissue heating, accelerated enzyme activity, muscle relaxation, pain reduction, and an enhancement of natural healing processes. Current techniques of ultrasonic contact medical physiotherapy are limited by the necessity of providing a direct contact interface between the ultrasonic transducer and the tissue to maintain an effective transmission of the ultrasonic waves, which subsequently results in acoustic burns on the target's surface. The technique is further limited by the fact that it can only produce a physical change in the area to which it is applied. Because this method affects a limited area, it is not feasible to use for conditions such as viral infections that affect cells throughout the entire body. Therefore, a need exists for an apparatus and methods that can destroy a virus and affect the whole body.

### SUMMARY OF THE INVENTION

The present invention is directed towards an apparatus and methods for the treatment of humans and birds infected with an avian influenza virus. Apparatus and methods in accordance with the present invention may meet the above-mentioned needs and also provide additional advantages and improvements that will be recognized by those skilled in the art upon review of the present disclosure, including but not limited to similar apparatus and methods for treating other infections and infectious agents.

The present invention delivers ultrasound waves to the target (an animal or human body) to destroy viruses and bacteria cells, to increase the blood flow, to enhance breathing ability, and to stimulate the immune system.

The present invention consists of an ultrasonic generator, a transducer, a sonicating horn, and a specially designed ultrasound tip that is able to deliver ultrasonic energy from the radiation surface to the target animal or human without contacting the target either through an air/gas medium or through a spray, or by contacting the target with or without a coupling medium such as a liquid, gel, gel pad, etc.

The method of the invention comprises producing and delivering ultrasonic energy to the target animal or human body without contacting the target through an air/gas medium or through a spray, or by contacting the target with or without a coupling medium such as a liquid, gel, gel pad, etc. The ultrasonic waves delivered through an air/gas medium to the body destroys viruses, kills bacteria cells and other infectious agents, increases the blood flow, and boosts the immune system. The ultrasound waves delivered to the body through the spray also kills viruses, destroys bacteria cells and other infectious agents, and boosts the immune system. Furthermore, the ultrasonic spray, because of the mechanical and vibration energy of the particles delivered to the body, increases the blood flow more effectively; it also increases the breathing ability by delivering drugs, antibiotics, etc to the lungs through the mouth of the target. Delivery of the ultrasound waves through the liquid coupling medium delivers an increased level of the ultrasound energy in comparison with the delivery through an air/gas medium or through a spray.

Ultrasonic energy directed to the body through an air/gas medium or a spray only provides an effect on the area to which it is locally directed; because of this, the ultrasonic transducer and tip in the present invention must be moved around the body to cover and sonicate the entire animal or human body. The most recommended area to cover, however, is the head and mouth because of the concentration of nerves in this region; furthermore, other recommended areas to cover are the abdomen, the back, the chest, and the thighs of the body.

This method of delivering ultrasonic energy to the target body from a distance has an advantage because other delivery methods are limited by requiring contact to a small area, and such contact can cause mechanical and acoustical damage or burns to the contacted tissue.

The invention is related to the special design of the ultrasonic tip, which allows for delivery of an increased level of ultrasonic energy to the body, and will be described below in detail.

### BRIEF DESCRIPTION OF THE DRAWINGS

FIG. 1 is a perspective view of an ultrasound virus treatment system for use according to the invention.

FIG. 2 is a cross-sectional view of an ultrasound virus treatment system for use according to the invention.

FIG. 3 is a detailed partial extended cross-sectional view of the distal end of an ultrasound virus treatment system with a special design tip that has both concave and convex curvatures.

FIG. 4 is a detailed partial extended cross-sectional view of the distal end of an ultrasound virus treatment system with a special design ultrasound tip that has both concave and convex curvatures, and the view of the acoustic energy as it emanates off of the tip.

FIG. 5 is a partial cross-sectional view of the sonicating horn section of an ultrasound virus treatment system that has an ultrasound tip with a concave curvature.

FIG. 6 is a detailed partial extended cross-sectional view of the distal end of an ultrasound virus treatment system with a concave-shaped ultrasound tip.

FIG. 7 is a detailed partial extended cross-sectional view of the distal end of an ultrasound virus treatment system with a concave-shaped ultrasound tip, and a view of the acoustic energy as it emanates off of the ultrasound tip.

FIG. 8 is a partial cross-sectional view of the sonicating horn section of an ultrasound virus treatment system that has a cone-shaped ultrasound tip.

FIG. 9 is a partial cross-sectional view of the distal end of an ultrasound virus treatment system with a cone-shaped ultrasound tip.

FIG. 10 is a partial cross-sectional view of the distal end of an ultrasound virus treatment system with a cone-shaped ultrasound tip, and a view of the ultrasound energy as it emanates off of the ultrasound tip.

FIG. 11 are front-views of a circular-shaped peripheral boundary of an ultrasound tip.

### DETAILED DESCRIPTION OF THE INVENTION

The present invention is a method and device that uses ultrasonic wave energy to treat humans and birds infected with an avian influenza virus. The device comprises a power generator, a hand-piece having a transducer, a sonicating horn, a specially designed ultrasound tip, a supply port, and a tube for delivering a liquid to the radiation surface of the ultrasound tip. Preferred embodiments of the present invention in the context of an apparatus and methods are illustrated in the figures and described in detail below.

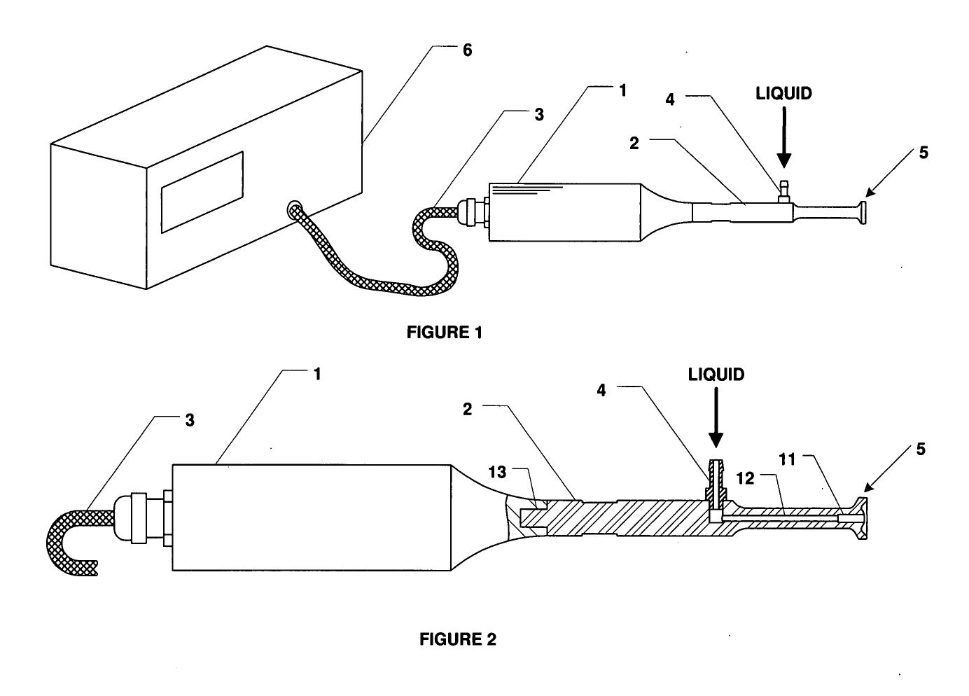

The ultrasound virus treatment system shown in FIG. 1 comprises a connection to a power generator 6 that supplies power via the power cable 3 to the ultrasonic transducer 1, which is connected to the sonicating horn 2. The liquid supply port 4 delivers a liquid the treatment system that exits out of the ultrasound tip 5. These sections are shown in detail in FIG. 2.

FIG. 2 is a cross-sectional schematic representation of the ultrasound virus treatment system shown in FIG. 1. The system comprises a connection to a power generator 3 that supplies power to the ultrasonic transducer 1, which is connected by a mechanical interface 13 to the sonicating horn 2. Examples of a mechanical interface include, but are not limited to, threading or bonding. The preferred embodiment contains a mechanical interface, but alternative embodiments could be used without a mechanical interface. The liquid supply port 4 delivers a liquid to the fluid conduit 12. The liquid is induced through the fluid conduit 12 by the sonicating horn 2 towards the transitional exit chamber 11 and out of the ultrasound tip 5.

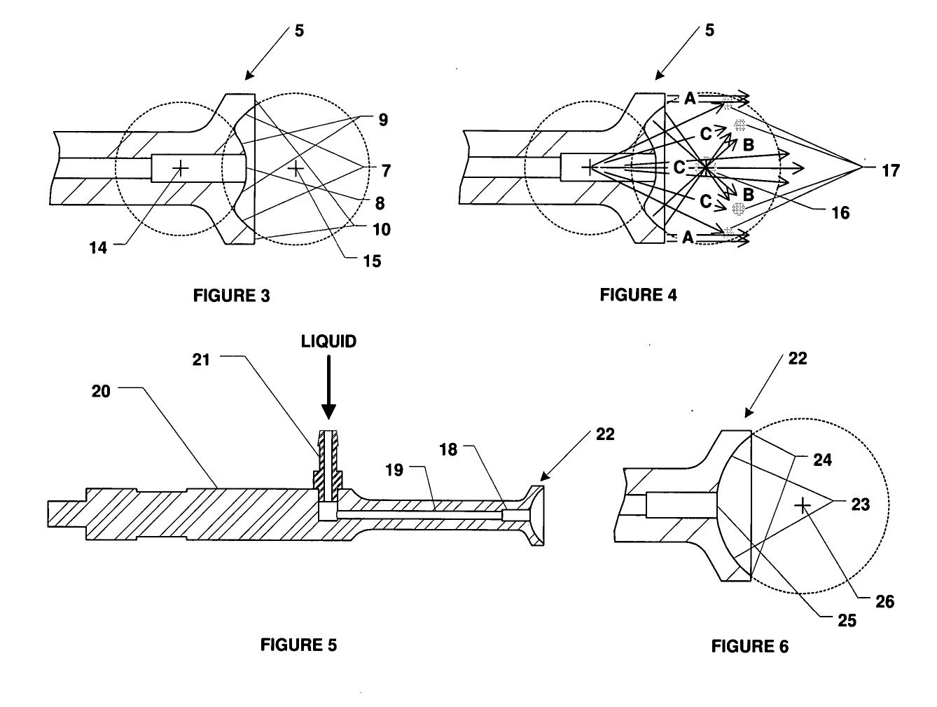

FIG. 3 is a detailed cross-sectional schematic representation of the ultrasound tip 5 shown in FIG. 2. The ultrasound tip is a special design—there is a distal planar end 10 that is perpendicular to the axis of the orifice 8, a convex curvature 9 at the opening of the orifice 8, and a concave curvature 7 that connects the convex curvature 9 to the planar end 10. The centers of the convex 9 and concave 7 curvatures are located at 14 and 15, respectively. This design is the preferred embodiment of the specially-designed ultrasound tip. Alternative embodiments include the planar end 10 in a different format including, but not limited to, a different angle, a different radius, or removed altogether.

FIG. 4 is a detailed cross-sectional schematic representation of the disbursement of the acoustic energy as it emanates from the specially designed ultrasound tip 5 from FIG. 3. Acoustic energy is directed horizontally outwards A from the planar end 10. Acoustic energy is also directed slanted inwards B from the concave curvature 7. Acoustic energy is also directed both horizontally and slanted outwards C from the convex curvature 9. There are local maximum focal points 17 where there is an increased level of acoustic energy resulting from the intersection of acoustic waves. There is also an absolute max focal point 16 where the acoustic energy B intersects with the acoustic energy C at the center point of the concave curvature 15—this contains the highest level of acoustic energy. This focal point 16 generates a level of acoustic energy that is greater than the level produced by a traditional ultrasound tip.

FIG. 5 is a cross-sectional schematic representation of the sonicating horn section with a concave-shaped ultrasound tip 22. This embodiment of an ultrasound virus treatment system also includes a sonicating horn 20 with a liquid supply port 21 that delivers a liquid to the fluid conduit 19. The liquid is induced through the fluid conduit 19 by the sonicating horn 20 towards the transitional exit chamber 18 and out of the ultrasound tip 22.

FIG. 6 is a detailed cross-sectional schematic representation of the concave-shaped ultrasound tip 22 shown in FIG. 5. The ultrasound tip 22 contains a concave curvature 23 that is connected to the distal planar end 24 that is perpendicular to the axis of the orifice 25. The center point of the concave curvature is located at 26. This design is the preferred embodiment of a concave-shaped ultrasound tip. Alternative embodiments include the planar end 24 in a different format including, but not limited to, a different angle, a different radius, or removed altogether.

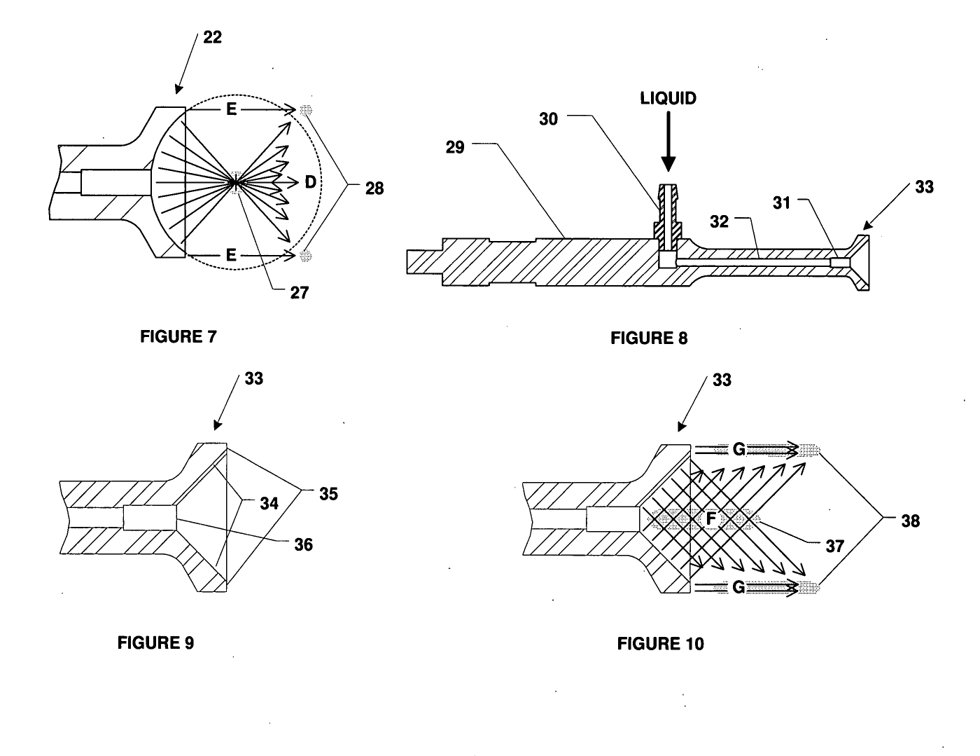

FIG. 7 is a detailed cross-sectional schematic representation of the disbursement of the acoustic energy as it emanates from the concave-shaped ultrasound tip 22 from FIG. 6. This concave shape results in acoustic energy D being directed inwards as it radiates off of the concave curvature 23 of the ultrasound tip 22. The acoustic energy D from the concave curvature 23 intersects to form a focal point 27 at the center point of the concave curvature 26 that generates an increased level of ultrasonic energy. The level of acoustic energy in this focal point 27 is not as high as the level of acoustic energy created at the absolute max focal point 16 from the convex-concave special design tip 5 shown in FIG. 4. There are also local maximum points 28 where acoustic energy D from the concave curvature 23 intersects with acoustic energy E from the planar end 24. These local maximum points 28 do not generate as high of a level of acoustic energy as the focal point 27.

FIG. 8 is a schematic representation of the sonicating horn section with a cone-shaped ultrasound tip 33. This embodiment of an ultrasound virus treatment system also includes a sonicating horn 29 with a liquid supply port 30 that delivers liquid to the fluid conduit 32. The liquid is induced through the fluid conduit 32 by the sonicating horn 29 towards the transitional exit chamber 31 and out of the ultrasound tip 33.

FIG. 9 is a detailed cross-sectional schematic representation of the cone-shaped ultrasound tip 33 shown in FIG. 8. The ultrasound tip 33 contains a cone-shaped curvature 34 that is connected to a distal planar end 35 that is perpendicular to the axis of the orifice 36. This design is the preferred embodiment of a cone-shaped ultrasound tip. Alternative embodiments include the planar end 35 in a different format including, but not limited to, a different angle, a different radius, or removed altogether.

FIG. 10 is a schematic representation of the disbursement of the acoustic energy as it emanates from the cone-shaped ultrasound tip 33 shown in FIG. 9. Similar to the tip in FIG. 5, acoustic energy F is directed inwards as it radiates off of the cone-shaped curvature 34. The energy directed off of both sides intersects to create a stream of multiple intersection focal points 37 and an enhanced level of acoustic energy. This level of acoustic energy is not as high as the level of acoustic energy created from either the concave-shaped tip in FIG. 6 or from the convex-concave special design tip in FIG. 3. Acoustic energy G is also directed horizontally outwards from the planar end 35. A stream of local focal points 38 are created where acoustic energy G intersects with acoustic energy F. The level of acoustic energy in the focal points 37 is greater than the level of acoustic energy in the local focal points 38.

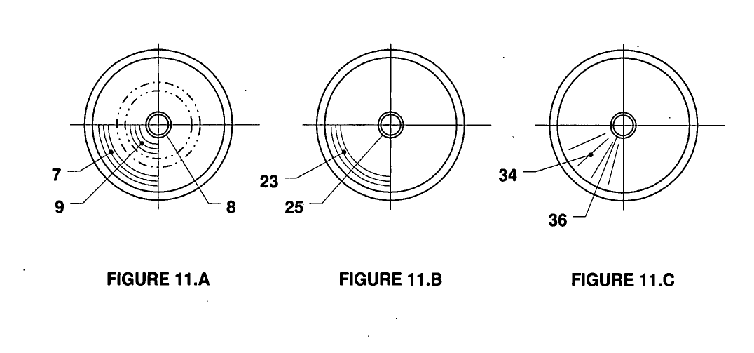

FIG. 11 are schematic representations of the front-view of the distal end of an ultrasound virus treatment system with an ultrasound tip that has a circular-shaped peripheral boundary. This front circular peripheral boundary can be utilized for each of the three ultrasound tip designs described above—the concave-convex special design tip in FIG. 2, the concave shaped tip in FIG. 5, and the cone shaped tip in FIG. 8, and the illustration of those shapes are included. FIG. 11a. is the front-view of the special design tip 5 from FIG. 3 and includes illustrations of the central orifice 8, the convex curvature 9, and the concave curvature 7. FIG. 11b. is the front-view of the concave-shaped ultrasound tip 22 from FIG. 6, and includes illustrations of the central orifice 25 and the concave curvature 23. FIG. 11c. is the front-view of the cone-shaped ultrasound tip 33 from FIG. 9, and includes illustrations of the central orifice 36 and the cone-shaped curvature 34.

The cross-section of the ultrasound tip can be curved, conical, the convex-concave design, or another similar shape. The most preferred shape is the convex-concave special design shown in FIG. 3 because it creates a focal point that allows for the generation of the highest level of ultrasonic energy. The distal front end peripheral boundary shape of the ultrasound tip may be circular as shown in FIG. 11, triangular, rectangular, or another similar shape. The most preferred front-end peripheral boundary is the circular shape shown in FIG. 11 because it creates the highest amount ultrasonic energy resulting from the intersection of the acoustic energy directed off of the ultrasonic tip.

The ultrasound virus treatment system shown in FIG. 1 delivers acoustic energy to an animal or human target without contacting the animal or human through an air/gas medium or through a spray, or by contacting the animal or human with or without a coupling medium such as a liquid, gel, gel pad, etc. The system is powered by an ultrasonic generator 6 that supplies electrical energy in an oscillating wave form. The transducer 1 converts that electrical energy into mechanical motion, which then induces a stress or displacement in the sonicating horn 2. That displacement in the sonicating horn 2 causes pressure and produces acoustic waves. A liquid is inserted into the fluid supply port 4. The liquid is pushed through the fluid conduit 12 by the pressure in the sonicating horn 2 and is released out of the central orifice 8. At the same time, the acoustic waves travel through the sonicating horn 2 and radiate off of the ultrasound tip 5. The acoustic energy is delivered to the animal or human body as it travels through an air/gas medium or through the spray, or as it travels directly to the target animal or human through contact with or without a coupling medium.

The design of this apparatus allows for a level of acoustic energy to be delivered to an animal or human that is higher than the level of acoustic energy provided by a traditional ultrasound device and tip. This level of acoustic energy delivered has an intensity capable of providing a multitude of therapeutic benefits. Depending upon the level of energy and the frequency of the waves, the acoustic energy can provide therapeutic effects such as killing viruses, destroying bacteria cells or other infectious agents, increasing blood flow, or stimulating the immune system. When used in conjunction with the delivery of a medicament to the animal or human's mouth to be absorbed into its lungs, the acoustic energy can more effectively increase the animal or human's breathing ability because it can deliver a smaller particle size to the target. Finally, the methods of delivering ultrasound energy through an air/gas medium, through a spray, or through a coupling medium prevent acoustic burns on the animal or human because the ultrasound tip 5 does not contact the body's surface.

Healthy cells and tissue have a natural ultrasonic frequency within which they resonate. Viruses, bacteria cells, and other infectious agents, however, have a different natural resonating frequency ranges. If the proper level of acoustic energy is delivered to an animal or human, the acoustic energy has the capability of disrupting viruses, bacteria cells, and other infectious agents at their respective resonating frequencies, therefore resulting in their destruction. At the same time, because of the difference in the natural resonance ranges, the frequency that destroys viruses, bacteria, and other infectious agents will leave healthy cells and tissue unharmed. Furthermore, the proper level of acoustic energy has the capability of stimulating the live animal or human's immune system as well as increasing its blood flow.

An important aspect of this invention is that it allows for treatment of the entire body of an animal or human. Because a virus infects cells through the entire body, it is not feasible to treat a single spot on the body like typical wound treatment methods. In order to destroy a virus, acoustic energy must be delivered to the whole body. The non-contact delivery method has an advantage because the transducer can be easily moved around to sonicate the entire the animal or human body, thus allowing for destruction all viruses within the body. The most recommended area to cover is the mouth and head because of the concentration of nerve cells in this region; furthermore, other recommended areas to cover are the abdomen, the back, the chest, and the thighs of the body. The most recommended method of treatment includes using multiple ultrasound delivery sources/transducers to sonicate different body parts at the same time. For humans, the recommended treatment method is to utilize multiple delivery sources/transducers to sonicate the body while the human is lying on a bed or sitting in a chair. For animals, the recommended treatment method is to mount multiple ultrasound delivery sources/transducers in a tunnel apparatus through which animals can be fed in order to treat multiple animals at once. These are the recommended treatment methods—other treatment methods can be similarly effective.

Preferably, the amplitude achieved by the acoustic energy is at least 3 microns or greater. Preferably, the frequency used is in the range of 20 kHz-60 MHz, wherein a preferred range is 20 kHz-100 kHz, and the most preferred value is 30 kHz. The recommended regimen to operate the ultrasound avian influenza treatment apparatus is to modulate the acoustic frequencies to cover broad ranges while treating an animal or human.

The method and device to treat avian influenza must be based on ultrasound and different energy sources such as ultrasound laser, magnetic field, infrared, microwaves, ultraviolet, RF, or cold or hot plasma energy, etc.

Although specific embodiments and methods of use have been illustrated and described herein, it will be appreciated by those of ordinary skill in the art that any arrangement that is calculated to achieve the same purpose may be substituted for the specific embodiments and methods shown. It is to be understood that the above description is intended to be illustrative and not restrictive. Combinations of the above embodiments and other embodiments as well as combinations of the above methods of use and other methods of use will be apparent to those having skill in the art upon review of the present disclosure. The scope of the present invention should be determined with reference to the appended claims, along with the full scope of equivalents to which such claims are entitled.

I claim:

1) A method for the treatment of avian influenza with ultrasound as well as the ultrasound treatment of other viruses, bacteria, and infectious agents such as human influenza, hepatitis C, etc., comprising the steps of:
a) delivering ultrasonic energy without contacting the target (an animal or human body) through an air/gas medium or through a spray, or by contacting the target with or without a coupling medium such as a liquid, gel, gel pad, etc., wherein the ultrasonic energy has an intensity capable of penetrating the body's surface to provide a therapeutic effect such as destroying viruses, bacteria cells, or other infectious agents, increasing blood flow, or stimulating the immune system, etc.;
b) sonicating and energizing the medicament,
c) delivering the medicament droplets through the mouth of the animal or human and into its lungs through an ultrasonic spray to provide a therapeutic effect.

2) The method according to claim 1, further including the step of generating the ultrasonic energy with particular ultrasound parameters indicative of an intensity capable of achieving a therapeutic effect.

3) The method according to claim 1, wherein the particular amplitude is at least 3 microns.

4) The method according to claim 1, wherein the frequency is in the range of 20 kHz-60 MHz.

5) The method according to claim 1, wherein the preferred frequency is in the range of 20 kHz-100 kHz.

6) The method according to claim 1, wherein the most preferred frequency value is 30 kHz.

7) The method according to claim 1, wherein the steps of the method are included in a series of treatments wherein another treatment of the series of treatments is selected from the group consisting of: delivering ultrasonic energy to the body, wherein the ultrasonic energy has an intensity capable of penetrating the body to provide a therapeutic effect to the body; sonicating the medicament and delivering the medicament droplets through the mouth of the animal or human and into its lungs through an ultrasonic spray to provide a therapeutic effect; the treatment including the steps of the method of the invention, wherein a different medicament is utilized.

8) The method according to claim 1, wherein the medicament is either: immunoglobulin, gamma globulin, interferon, an antibiotic, an ointment, cream, gel, liquid, salve, oil, saline solution, distilled, non-distilled and/or boiled water, powder, spray, antibacterial agent, antiseptic agent, insulin, analgesic agent, conditioner, surfactant, emollient, or other active ingredient or agent, etc.

9) The method according to claim 1, wherein the medicament is applied either before, after, or during delivery of the ultrasonic energy.

10) The method according to claim 1, wherein the step of delivering ultrasonic energy includes the step of providing means for delivering the ultrasonic energy through an air/gas medium or through a spray without contacting the animal or human target.

11) The method according to claim 1, wherein the step of delivering ultrasonic energy includes the step of providing means for delivering the ultrasonic energy by contacting the animal or human target with or without a coupling medium such as a liquid, gel, gel pad, etc.

12) The method according to claim 1, wherein the therapeutic effect provided is the destruction of viruses, bacteria cells, or other infectious agents, the increase blood flow in the body, the stimulation of the immune system, or the enhancement of breathing ability, etc.

13) An apparatus for the treatment of avian influenza with ultrasound as well as the ultrasound treatment of other viruses, bacteria, or infectious agents such as human influenza, hepatitis C, etc., comprising:
a) a generator and a transducer for generating ultrasonic energy;
b) wherein the transducer delivers the ultrasonic energy to the body or delivers a medicament to the lungs through the mouth; and
c) wherein the ultrasonic energy has an intensity capable of applying acoustic energy and penetrating the animal or human's body to provide a therapeutic effect such as destroying viruses, bacteria cells, or other infectious agents, increasing blood flow, or stimulating the immune system, etc., or of sonicating and energizing the medicament and delivering the medicament droplets through the mouth of the animal or human and into its lungs through an ultrasonic spray to provide a therapeutic effect

14) The apparatus according to claim 13, wherein the generator and transducer generate the acoustic energy with particular ultrasound parameters indicative of an intensity capable of achieving a therapeutic effect.

15) The apparatus according to claim 13, wherein the particular amplitude is at least 3 microns.

16) The apparatus according to claim 13, wherein the frequency is in the range of 20 kHz-60 MHz.

17) The apparatus according to claim 13, wherein the preferred frequency is in the range of 20 kHz-100 kHz.

18) The apparatus according to claim 13, wherein the most preferred frequency value is 30 kHz.

19) The apparatus according to claim 13, wherein the transducer includes a radiation surface having a surface area dimensioned/constructed for achieving delivery of the ultrasonic energy to the animal or human with an intensity capable of achieving a therapeutic effect.

20) The apparatus according to claim 13, wherein the transducer includes a radiation surface where the shape of the curvature of the radiation surface is either concave, conical, the concave-convex special design, or another comparable shape intended to achieve delivery of the ultrasonic energy to the animal or human with an intensity capable of achieving a therapeutic effect.

21) The apparatus according to claim 13, wherein the shape of the peripheral boundary of the radiation surface is either circular, polygonal, or another comparable shape intended to achieve delivery of the ultrasonic energy to the animal or human with an intensity capable of achieving a therapeutic effect.

22) The apparatus according to claim 13, wherein the transducer includes a radiation surface; a selection is made of a size and of a surface area of the radiation surface, a shape of the peripheral boundary of the radiation surface that is circular, polygonal, or another comparable shape, and a shape of the curvature of the radiation surface that is either concave, conical, the concave-convex special design, or another comparable shape; and the particular ultrasound parameters of the generated ultrasonic energy for achieving delivery of ultrasonic energy to the animal or human target with an intensity capable of achieving a therapeutic effect.

23) The apparatus according to claim 13, wherein the step of delivering ultrasonic energy includes the step of providing means for delivering the ultrasonic energy through an air/gas medium or through a spray without contacting the animal or human target.

24) The apparatus according to claim 13, wherein the step of delivering ultrasonic energy includes the step of providing means for delivering the ultrasonic energy by contacting the animal or human target with or without a coupling medium such as liquid, gel, gel pad, etc.

25) The apparatus according to claim 13, wherein the transducer is driven by a continuous, pulsed, or modulated frequency and wherein the driving wave form of the transducer is selected from the group consisting of sinusoidal, rectangular, trapezoidal and triangular wave forms.

26) The apparatus according to claim 13, wherein the therapeutic effect provided is the destruction of viruses, bacteria cells, or other infectious agents, the increase blood flow in the body, the stimulation of the immune system, or the enhancement of breathing ability, etc.

27) The apparatus according to claim 13, wherein the medicament delivered by ultrasound is either: immunoglobulin, gamma globulin, interferon, an antibiotic, an ointment, cream, gel, liquid, salve, oil, saline solution, distilled, non-distilled and/or boiled water, powder, spray, antibacterial agent, antiseptic agent, insulin, analgesic agent, conditioner, surfactant, emollient, or other active ingredient or agent, etc.

28) The apparatus according to claim 13, wherein the medicament is applied either before, after, or during delivery of the ultrasonic energy.
